(19) United States
(12) Patent Application Publication
(10) Pub. No.: US 2020/0256749 A1
(43) Pub. Date: Aug. 13, 2020

(54) TWO-WHEEL COMPONENT WITH A MEASURING DEVICE

(71) Applicant: DT SWISS INC., Grand Junction, CO (US)

(72) Inventors: Martin WALTHERT, Aarberg (CH); Simon HUGENTOBLER, Liebefeld (CH); Jean-Paul Victor BALLARD, Thalwil (CH); Seamus MULLARKEY, Tann (CH); Jonas Gretar JONASSON, Thalwil (CH)

(21) Appl. No.: 16/785,334

(22) Filed: Feb. 7, 2020

(30) Foreign Application Priority Data
Feb. 9, 2019 (DE) 102019103232.6

(51) Int. Cl. G01L 3/24; G01L 1/22; B62J 45/40; A61B 5/11

(52) U.S. Cl. CPC G01L 3/24; A61B 5/1118; B62J 45/40; G01L 1/22

(57) ABSTRACT

A bicycle component with a measuring device with a housing and a measuring probe connected therewith, including a probe body having an exterior opening. The exterior opening is connected through an air guide with a barometric pressure sensor disposed remote from the exterior opening. The air guide includes at least two air ducts and an internal chamber that is connected with two air ducts. One of the air ducts is configured as a supply duct and begins at the exterior opening. The other of the air ducts serves as a sensor duct and connects the internal chamber with the barometric pressure sensor.

The present invention relates to a bicycle component with a measuring device including at least one measuring probe and at least one barometric pressure sensor for capturing a measure of the ambient pressure at a bicycle in which the bicycle component is mounted. For example, the air pressure signal of a barometric pressure sensor allows to compute the current elevation of the bicycle respectively to capture in particular elevation changes during the ride. These measurements may be employed for a variety of purposes. The data may for example be stored for later evaluation. These and further measurement data also allow to determine the air drag of the bicycle including the rider sitting thereon during riding.

Bicycle components mounted to bicycles are exposed to a great variety of weather conditions. Thus, initially good weather may change during a tour or a race and bring down sudden precipitations. Then the bicycle components employed with a bicycle must either be insensitive to moisture or structured so that rain showers do not damage the bicycle components installed. Energy demand is another significant aspect. Thus, the measuring apparatus of a bicycle cannot be elaborately temperature-controlled and cooled or heated, to provide the same results in any and all ambient conditions.

It is therefore the object of the present invention to provide a bicycle component with a measuring device which is largely insensitive to moisture influences during riding or also in standstill. The measuring device is in particular intended to be insensitive to other contamination.

This object is solved by a bicycle component having the features of claim 1. Preferred specific embodiments of the bicycle component according to the invention are subject matter of the subclaims. Further advantages and features of the present invention can be taken from the general description and the description of the exemplary embodiments.

A bicycle component according to the invention comprises in particular a housing and at least one measuring device and at least one measuring probe connected with the housing. The measuring probe comprises at least one probe body, which is particularly preferably unheated and includes at least one exterior opening (or multiple exterior openings, in particular at the probe body) wherein the exterior opening is connected by an in particular internal air guide with at least one barometric pressure sensor or multiple barometric pressure sensors disposed remote from the exterior opening. The (internal) air guide comprises at least two or more air ducts and comprises at least one in particular internal chamber, which is connected with at least two air ducts in particular through one or multiple inlet(s) and outlet(s). One of the air ducts is configured as a supply duct and begins at the exterior opening (of the probe body). Another air duct serves as a sensor duct and connects the internal chamber with the barometric pressure sensor. The connection may be indirect or direct. At least one of the air ducts or at least two of the air ducts or all of the air ducts have a minimum clear diameter of less than 2.5 mm.

The bicycle component according to the invention has many advantages. A considerable advantage of the bicycle component according to the invention is achieved by the internal air guide which comprises two or more air ducts, one of the air ducts extending from the exterior surface of the probe body up to an internal chamber and wherein the internal chamber is coupled or connected with the barometric pressure sensor through at least one other air duct or sensor duct. Thus the exterior surface of the probe body is connected with the barometric pressure sensors only through the air guide inside the probe body. The probe body is in particular not temperature-controlled and preferably not heated, nor is it cooled. This means that the measuring probe is physically configured so as to enable efficient, repeatable measurements in any and all weather situations. To this end, the structure prohibits the entry of water. It is advantageously achieved by air ducts showing reduced diameters.

Barometric pressure sensors are as a rule hygroscopic and should not be exposed to direct contact with water. The bicycle component according to the invention sufficiently protects barometric pressure sensors. The internal chamber enables absorption of any penetrating water, so that any water or other substances are virtually excluded from penetrating into the supply duct up to the barometric pressure sensors.

A minimum clear diameter of at least one (or at least two) of the air ducts is in particular less than 2.0 mm, in particular less than 1.5 mm. Particularly preferably the minimum clear diameter is less than 1.25 mm. Particularly preferably the minimum clear diameter is between 0.25 mm and 1.5 mm and particularly preferably between 0.75 mm and 1.25 mm.

The minimum clear diameter is not understood to mean the diameter of a bottleneck but a clear diameter extending over a length of a minimum of 10% of the length of an air duct. A clear diameter changing e.g. continuously from one end to the other means that the minimum clear diameter is the diameter averaged over the first section of 10% of the length. Given a constant cross section, the minimum clear diameter equals what is the local diameter.

Particularly preferably the dimension of at least one (or at least two) of the air ducts near the outer opening is less than 1.5 mm and in particular less than 1.25 mm. Particularly preferably the minimum clear diameter is less than 1.25 mm. Particularly preferably the dimension at the outer opening is between 0.25 mm and 1.25 mm and particularly preferably between 0.5 mm and 1.0 mm. Small diameters or dimensions at the outer opening of the air ducts offer reliable protection against the entry of water into the interior and therefore provide reliability of function.

Preferably the dimension at the outer opening is smaller than an average or typical diameter of the pertaining air duct. The typical diameter is the representative diameter given over the majority of the length of an air duct.

The dimensions are responsible for largely prohibiting the entry of water into the measuring probe at all times.

Preferably at least one takeup space is configured in an internal chamber for any (still) penetrating water. This allows to reliably collect any water that may have penetrated through the exterior opening of the probe body and the barometric pressure sensor is sufficiently protected.

The takeup space is preferably configured well-shaped. In particular, both the inlet and the outlet of the takeup space are spaced apart from the floor of the takeup space so as to provide a suitable reservoir for any entering water.

In all the configurations it is possible and also preferred for the internal chamber or at least one internal chamber to comprise at least one partition wall. The internal chamber is subdivided into at least two chamber sections by a partition wall. The two chamber sections are preferably interconnected through at least one connecting opening. The connecting opening of the chamber sections is in particular configured in the partition wall. It is also possible for the connecting opening to be configured as a duct. As a rule, a small opening, which serves as a connecting opening in the partition wall, is simpler and therefore preferred.

When an internal chamber is subdivided into two chamber sections by a partition wall, different takeup spaces are provided which further inhibits the entry of water.

In particular, in use as intended the connecting opening is disposed spaced apart from the bottom end of the internal chamber. This forms a collecting space for any entered water in the lower region. It is possible and preferred to configure the probe body with several internal chambers or multiple chamber sections interconnected via air ducts or intermediate ducts. The connecting openings are in particular disposed offset to one another in multiple chamber sections arranged in series, so that further entry of water is inhibited.

Preferably, the air guide is configured with at least one type of labyrinth seal. Such a labyrinth seal may be formed for example by multiple chambers or chamber sections arranged in series with the connecting openings between the chamber sections disposed offset laterally and/or in height (when installed as intended). The multitude of the connecting openings or all of the connecting openings or gates of the intermediate ducts are preferably disposed spaced apart from the bottom of the pertaining chamber. This in particular achieves an air guide configuration in a zigzag layout. A takeup space for any entering water is preferably configured in the bottom regions of the pertaining chambers. This largely prevents the entry of water up to the sensor duct which finally leads up to the barometric pressure sensor.

In all the configurations, it is possible for the labyrinth seal to comprise two or more internal chambers and in-between, air ducts and/or two or more chamber sections and disposed in-between, connecting openings or ducts.

The bicycle component is preferably configured such that the feed unit and all the chambers offer ease of cleaning (e.g. by purging or the like). The bicycle component preferably offers ease of maintenance.

Preferably, the housing and/or the probe body is configured aerodynamically. The probe body is preferably elongated and shows a substantially rotationally symmetrical outer surface. An opening is preferably configured centrally at the front tip of the probe body, defining the beginning of the supply duct. It is also possible to have two or more openings configured in lateral regions of the tip of the probe body connected with one or more sensors through suitable air ducts.

It is preferred for the probe body to consist of at least one metal at least partially and in particular considerably or predominantly or nearly entirely or entirely.

Particularly preferably, at least a considerable part of the probe body is manufactured in particular integrally wherein the air guide is formed by 3D printing. Such a three-dimensional printing process allows efficient manufacturing of what is a complex probe body. The probe body may be configured seamless, showing in the interior an air guide comprising multiple air ducts and multiple chambers and chamber sections. The outer surface may be finished mechanically.

Preferably, the probe body shows at least two or three openings to which at least one barometric pressure sensor or two or more barometric pressure sensors or one barometric pressure sensor each are assigned.

The probe body is preferably connected with the housing. The probe body is preferably plugged on. The probe body is in particular detachably connected with the housing.

Particularly preferably, the at least one barometric pressure sensor is disposed in the housing. A suitable further sensor duct may be provided in the housing for connecting the barometric pressure sensor with the sensor duct.

Preferably, a computer is disposed in the housing. Further sensors may be disposed in the housing to allow capturing a wide variety of data.

Preferably, the dimensions of the exterior openings at the probe body and at the internal air guide are designed such that the resulting cross section of the air ducts (respectively the air guide geometry) is small enough for the water surface tension at the cross section of the air duct or the air ducts to withstand the force ensuing from the dynamic air pressure with air speeds of up to 20 m/s and in particular at least 30 m/s and preferably at least 35 m/s or up to 35 m/s. This prevents water from penetrating up to the barometric pressure sensors. A considerable advantage thereof is that the air volume already present in the air duct forms a type of air spring if water penetrates into the pertaining air duct. Since due to the water surface tension the water completely fills the duct cross section, penetrating water may only travel further into the duct against the spring force of the composing air behind the water plug. This is why the pressure increases along with the entry depth, while improved safeguarding is obtained at the same time.

In preferred specific embodiments, each of the internal chambers contains a takeup space or a volume in which any water entering the measuring probe through the exterior openings may collect. Preferably, substantially each internal chamber, or each internal chamber, is positioned such that the takeup space is remote from the outlet of the internal chamber in the direction of the barometric pressure sensor.

In use as intended, the supply duct is preferably configured ascending so that the exterior opening of the supply duct is disposed lower than the gate region of the supply duct into an internal chamber. Preferably, the outlet of the last internal chamber into the sensor duct is disposed on an elevated level, so that first all the preceding takeup spaces must fill with penetrating water before penetrated water passes through the sensor duct.

In all the configurations, it is preferred to fasten the bicycle component in a front region of the bicycle. The bicycle component may for example be directionally mounted to the handlebar so that the probe body always points in the current traveling direction.

Preferably, the bicycle component is as small and aerodynamic as possible, in particular so as to create minimal air drag both with frontal and lateral approach of air.

The bicycle component or further parts and sensors may serve to capture various data for determining the current driving power, the current traveling speed, the current path or road gradient, the current wind speed and wind direction. Various data allows conclusions about the current riding conditions. It is possible to derive the current aerodynamic drag while the bicycle is traveling, including a rider who may be sitting on it, to assist the rider in assessing his current position on the bicycle or to facilitate the use of various components.

The invention is used in particular with at least partially, or entirely, muscle-powered two-wheeled vehicles and in particular bicycles. Therefore, the term two-wheeled vehicle component may be continuously replaced by the term bicycle component. Its use is possible and preferably provided in particular with so-called “Light Electric Vehicles” (LEV), meaning electric vehicles having two or four wheels driven by a battery, fuel cell or hybrid drive, generally weighing less than 100 kg and preferably less than 80 kg and particularly preferably less than 50 kg or 30 kg. Particularly preferably the invention is used for use in open-top two-wheeled vehicles or bicycles. These are roof-less, two-wheeled vehicles.

The bicycle component comprises in particular an energy source such as a battery. Particularly preferably the bicycle component comprises a display and/or an interface with a display. Furthermore, it is possible for at least one humidity sensor to be comprised. In particular at least one temperature sensor may also be comprised. A humidity sensor and/or a temperature sensor may for example obtain the air density.

A two-wheeled vehicle and in particular a bicycle in the sense of the present invention preferably comprises two wheels on two different axles. In particular, in use as intended the two wheels are disposed (at least substantially or entirely) in tandem.

Further advantages and features can be taken from the exemplary embodiments which will be discussed below with reference to the enclosed figures.

The figures show in:

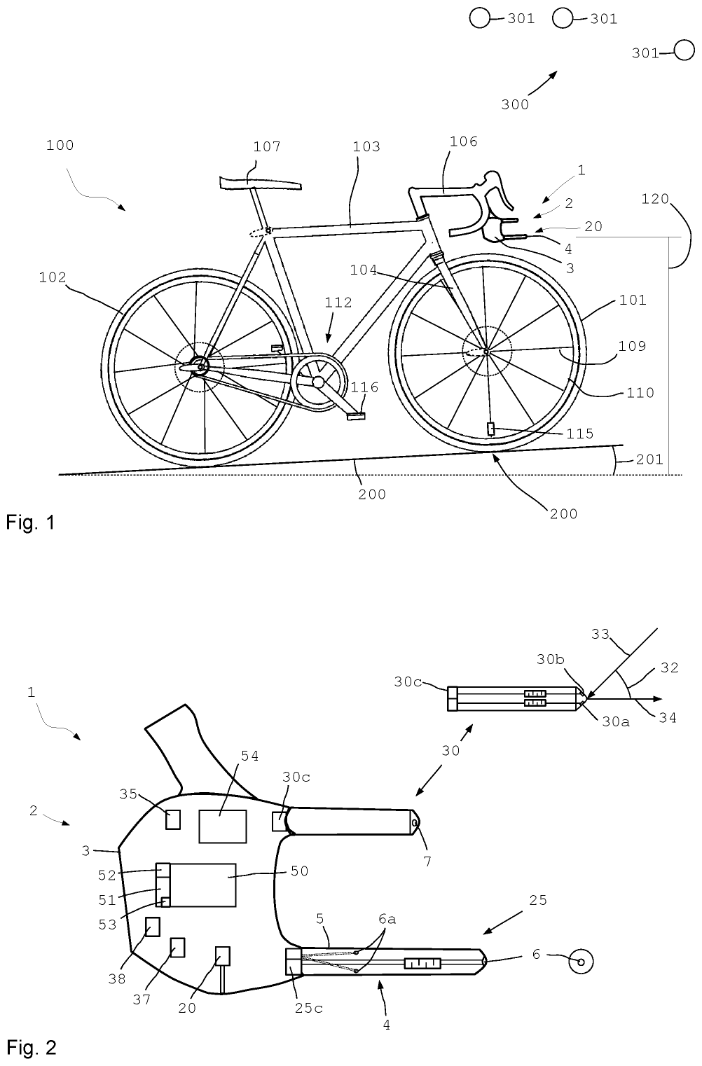

FIG. 1 a schematic side view of a racing bicycle on an ascending road including a bicycle component according to the invention;

FIG. 2 a bicycle component in a schematic side view;

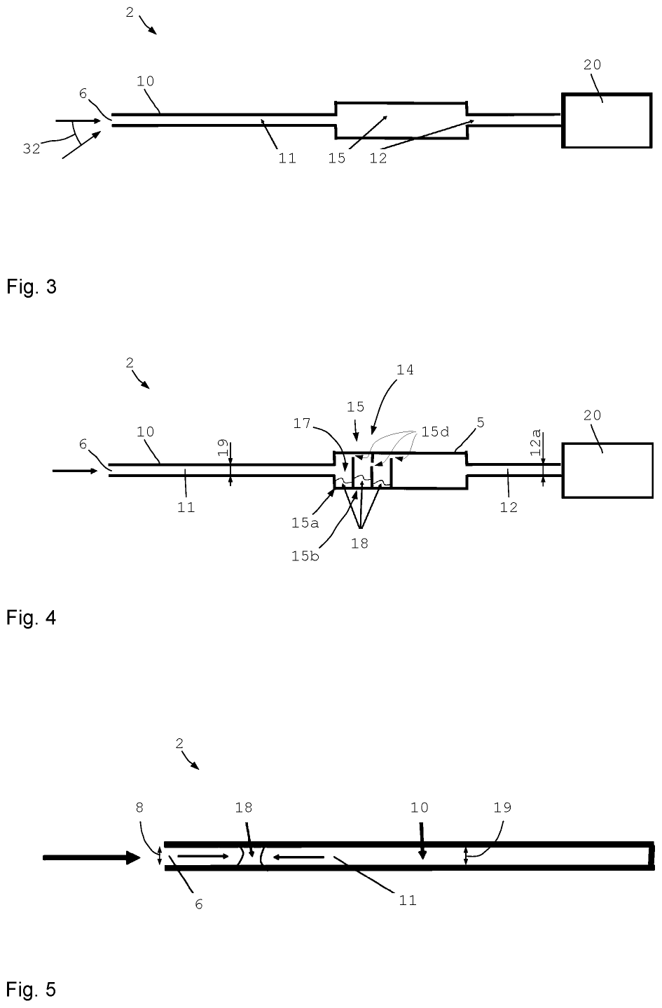

FIG. 3 a sectional diagrammatic drawing of a bicycle component;

FIG. 4 another sectional diagrammatic drawing of a bicycle component;

FIG. 5 a schematic detail of a bicycle component;

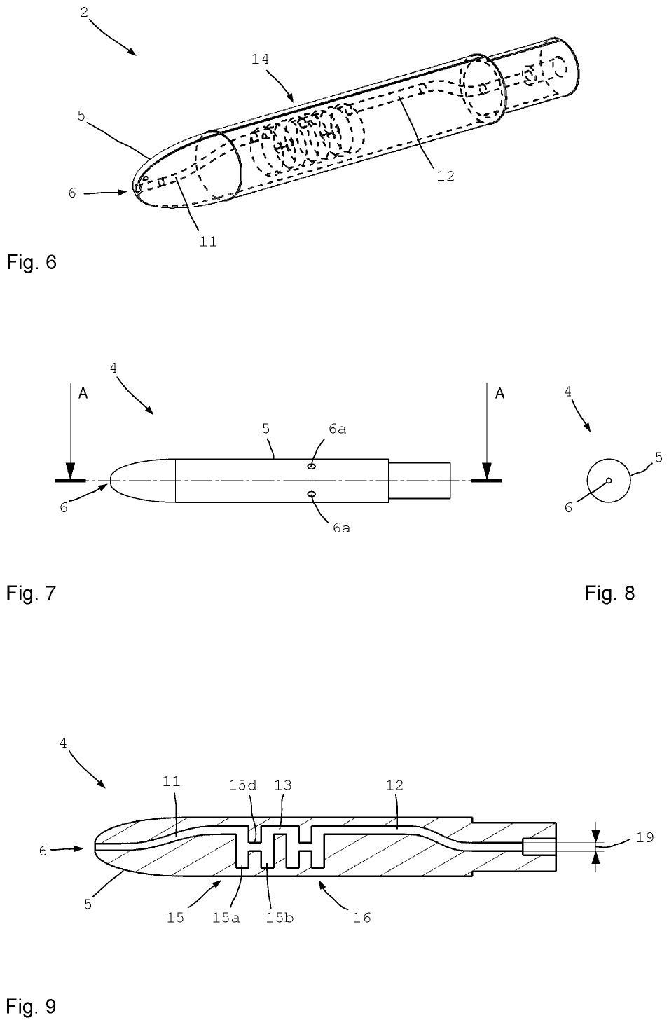

FIG. 6 a perspective illustration of a measuring probe of a bicycle component;

FIG. 7 the measuring probe according to FIG. 6 in a side view;

FIG. 8 a front view of FIG. 6;

FIG. 9 a section of FIG. 6;

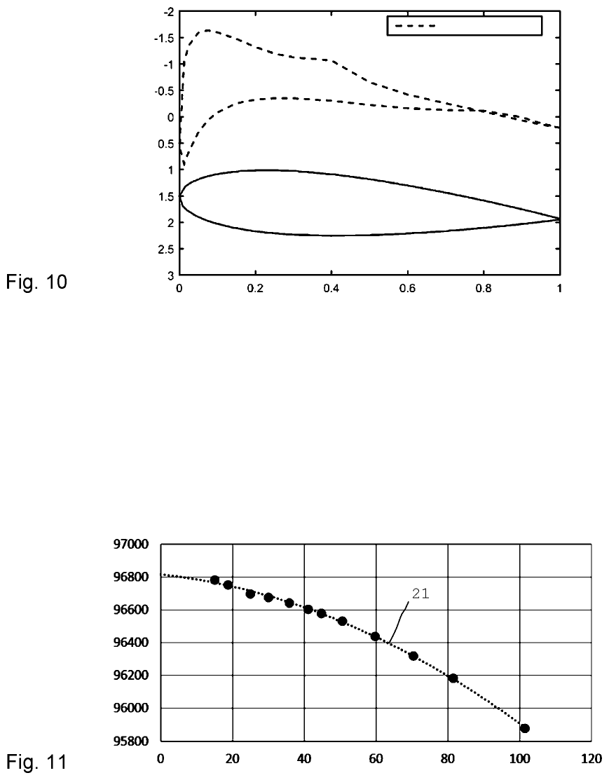

FIG. 10 an outline of the pressure coefficient on a surface of a body in the air stream;

FIG. 11 an outline of the absolute ambient pressure measured with a bicycle component over the air speed relative to the bicycle; and

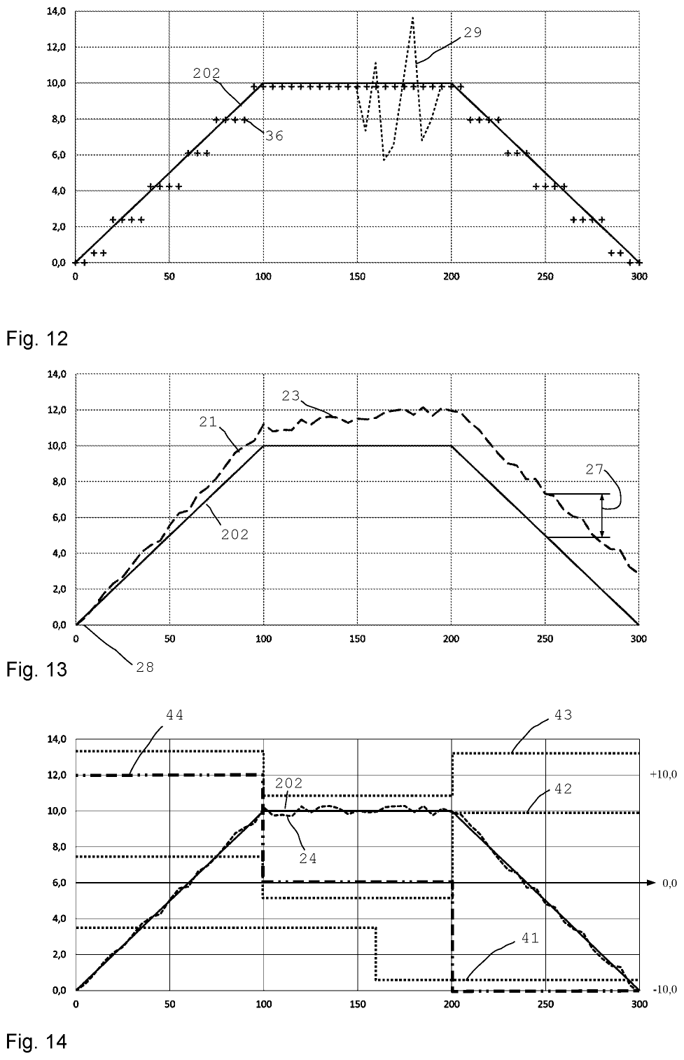

FIGS. 12 to 14 an elevation curve of a road over the track and values measured during riding on said track.

FIG. 1 illustrates a racing bicycle 100 wherein the invention may also be used in a mountainbike. The racing bicycle 100 comprises a front wheel 101 and a rear wheel 102. The two wheels 101, 102 are provided with spokes 109 and a rim 110. Conventional caliper brakes or other brakes such as disk brakes may be provided.

A bicycle 100 comprises a frame 103, a handlebar 106, a saddle 107 and a fork. A pedal crank 112 with pedals serves for driving. Optionally the pedal crank 112 and/or the wheels may be provided with an electrical auxiliary drive. The hubs of the wheels may be fastened to the frame e.g. by means of a through axle or a quick release.

The racing bicycle 100 illustrated in FIG. 1 travels uphill on a path or street 200. The gradient angle 201 indicates the present gradient. One or more speed sensors 115 serve to obtain the traveling speed of the racing bicycle 100 on the path 200. The speed may be obtained by way of spoke sensors or in the wheel itself and/or through satellite systems. Power or force sensors 116 at the pedals and/or pertaining sensors at the pedal crank and/or at the rear wheel hub serve to compute the driving power of the racing bicycle 100.

The bicycle component 1 with the measuring device 2 is directionally fastened to the handlebar 106 and/or the fork. The captured data can be evaluated, stored and processed in the bicycle component 1 or the measuring device 2 of the bicycle component 1 or in a separate (bicycle) computer.

The bicycle component 1 comprises a measuring device 2 and a housing 3 where one or more measuring probes 4 are disposed. The measuring probe 4 may be configured as a pitot tube and may capture a measure of the stagnation pressure at the front end of the measuring probe 4 or the probe body 5. An internal air guide allows to feed the total pressure to a barometric pressure sensor where it is captured. Taking into account the ambient pressure allows to derive the stagnation pressure. Preferably a differential pressure sensor is used for capturing a pressure difference between the total pressure at the front end and a lateral, local static pressure (local ambient pressure) on the measuring probe 4. It is also possible to use two absolute pressure transducers and to obtain their difference for computing the stagnation pressure.

FIG. 2 shows an enlarged illustration of the bicycle component 1 from FIG. 1 and schematically shows several components or parts of the bicycle component 1. The bicycle component 1 comprises a measuring device 2. The measuring probe 4 with the probe body 5 is disposed at the front end of the bicycle component 1 viewed in the traveling direction, to capture the stagnation pressure at the front end of the bicycle component 1 and thus near the front end of the bicycle 100. Positioning in the front region substantially avoids possible influences by further components of the racing bicycle 100.

At its front end the probe body 5 shows the outwardly opening 6. Through an air guide 10, which will be discussed in detail below, in the interior of the probe body 5, the opening 6 is connected with the schematically illustrated stagnation pressure sensor system 25. A front view is schematically illustrated on the right next to the bicycle component proper. The round probe body 5 with the central front opening 6 is identifiable.

The probe body 5 of the measuring probe 4 is elongated in shape and approximately cylindrical over a substantial part of its length. At least one hole 6a is configured spaced apart from the front end and presently in an approximately central section on the circumference. This hole 6a is connected with the stagnation pressure sensor system 25 on the side wall of the probe body 5. The central front opening 6 is likewise connected with the stagnation pressure sensor system 25.

The stagnation pressure sensor system 25 comprises a differential pressure sensor 25c, which captures a differential pressure between the openings 6 and 6a. Thus, a dynamic differential pressure is captured from which a stagnation pressure value or air pressure value is derived. A value for the local static pressure is captured via the openings 6a while the total pressure during the ride is captured through the opening 6. The differential pressure obtained with the differential pressure sensor 25c of the stagnation pressure sensor system 25 is a measure for the relative air speed streaming frontally onto the probe body.

A number of openings 6a are preferably evenly distributed over the circumference and interconnected inside the probe body 5 so that they capture an average static pressure. FIG. 2 exemplarily shows two openings 6a, each being disposed slightly above and approximately below the center line. The openings 6a may be interconnected in the longitudinal section of the openings 6a or may be connected with the differential pressure sensor 25c through separate ducts. Two or more and in particular three, four, five, six, seven or eight or more openings 6a may be (symmetrically) distributed over the circumference.

In the interior of the probe body 5 the indicated ducts are in particular configured in all the ducts so as to prevent water from penetrating up to the sensor.

The bicycle component 1 furthermore comprises a barometric pressure sensor 20 for capturing the ambient pressure. The barometric pressure sensor 20 for capturing the ambient pressure may be disposed in a number of positions of the measuring device 2. At any rate the barometric pressure sensor 20 should not also capture the total pressure which is captured by the stagnation pressure sensor 25 at the foremost tip of the measuring device 2.

For capturing the ambient pressure, the barometric pressure sensor 20 (also referred to as absolute pressure transducer) may for example be disposed inside the housing 3, specifically in a lower region of the housing 3 or in the rear region of the housing 3. It is also possible for the barometric pressure sensor 20 for capturing the ambient pressure to be disposed on a side surface or at the bottom face of the housing 3 or to include an inlet surface. At any rate the barometric pressure sensor 20 captures an air pressure signal 21 for the ambient pressure but not for some other pressure which might lie between the total pressure and the ambient pressure.

The bicycle component 1 illustrated in FIG. 2 furthermore comprises a satellite sensor 35 with which signals can be received from a satellite system 300 or its satellite 301 (see FIG. 1) to derive an elevation signal 36 in a known manner (FIG. 12). A humidity and/or temperature sensor 37 may be provided for determining the air humidity and/or air temperature, and may also be used for computing the air density of the ambient air. An acceleration sensor 38 serves to capture the accelerations of the racing bicycle 100.

By means of a computer 50 comprising a memory 51 and a data interface and in particular a network interface 52 the captured data may be processed, stored, and optionally transmitted to remote stations. The data interface may also comprise an antenna for receiving and/or emitting signals. Data can thus be optionally radio-transmitted.

The power source 54 may be a battery or an accumulator or another energy storage device to provide the energy required for the sensors, the memory and the computer. Energy supply through the bicycle is also conceivable.

A yaw sensor 30 comprises a differential pressure sensor 30c for capturing the differential pressure at the two openings 30a and 30b disposed at the front end of the yaw angle probe. The yaw angle probe is configured at its front end with two surfaces angled relative to one another (in particular perpendicular to the ground) and presently oriented at an angle of 90° to one another, and comprises the two openings 30a and 30b. A yaw angle 32 is derived from the measurement values.

The yaw sensor comprises a probe body similar to that of the stagnation pressure sensor system 25. Two separate air guides are configured in the interior of the probe body of the yaw sensor 30.

The front tip is provided with two openings 30a, 30b at angles relative to one another, in particular connected with a differential pressure sensor 30c or separate pressure sensors to derive a differential pressure.

To facilitate overview, the half of FIG. 2 on the right shows a top view of the probe body of the yaw sensor 30 from which it can be seen that the openings 30a, 30b of the differential pressure sensor 30c are oriented at angles to one another.

Thus it is possible to obtain from the traveling speed 34 and the captured values, the wind direction and the wind speed 33 relative to the movements of the bicycle 100. Said wind direction and wind speed 33 correspond to the wind blast to which the rider is exposed at the yaw angle 32.

It is also possible to provide two or optionally more surfaces on which to measure the air pressure disposed e.g. at angles to one another to derive a yaw angle 32 from the differences between the measurement values.

FIGS. 3 to 5 are schematic illustrations of a bicycle component 1 respectively a measuring device 2 with a measuring probe 4. FIG. 3 shows a simple example of a measuring probe 4 including a graphic illustration of one of the air guides 10.

For the sake of clarity, only one air guide 10 each is shown although the yaw sensor 25 or the stagnation pressure sensor system 30 for capturing the stagnation pressure preferably each comprise differential pressure sensors and two or more separate air guides 10. Various air guides 10 are separate from one another inside of a probe body, comprising separate chambers 15 and/or chamber sections and optionally partition walls 15c to prevent water and/or dirt from entering up to the pressure sensor or differential pressure sensor.

At the front end of the probe body 5 the outwardly opening 6 is formed which is followed by the air guide 10 and firstly the air duct 11 as a supply duct. The air duct 11 extends up to the chamber 15 which provides a takeup space for any entered water. In a preferred configuration a typical diameter 19 of the air duct 11 is approximately 1 mm (+/−20%). The narrow diameter already largely prohibits the entry of water.

The rear end of the chamber 15 is followed by the air duct 12 that is configured as a sensor duct and extends up to the barometric pressure sensor 20. The typical diameter 12a of the sensor duct 12 is also approximately 1 mm (+/−20%) in a preferred configuration. The structure of the air guide 10 and the narrow diameter of the air and sensor ducts ensure reliable protection of the barometric pressure sensor 20 against penetrating water.

Another contribution to protection against penetrating water is the fact that the outer opening 6 of the air duct 11 shows a dimension or diameter 8 (which is smaller still than the diameter of the air duct 11). The diameter 8 is about 20% smaller than the typical diameter 19 of the air duct 11. An outer opening 8, that is smaller still, achieves a still better protection against penetrating water.

This allows to omit thermal measures such as heating the probe body 5. The interior remains largely free from water in operation. However, at least in the region of the probe body 5 the bicycle should not be cleaned by means of a high pressure cleaner.

Firstly, the takeup space formed in the chamber 15 would have to fill up with water before water can enter the sensor duct 12. Due to the narrow dimensions and the water's surface tension any entering water forms a plug that tightly closes the duct and thus entraps the air volume present behind in the sensor duct 12. For water to penetrate further into the sensor duct 12 the entrapped air volume must to be compressed so that a counterforce acts against penetrating water. In this way, water is largely prevented from penetrating up to the barometric pressure sensor 20.

The sensors 25, 30 may be adapted or configured similarly to the illustration in FIGS. 3 to 9.

FIG. 4 shows a variant where the inner chamber 15 is subdivided into a number of chamber sections 15a, 15b etc. To this end, partition walls 15c are provided subdividing the chamber 15 in chamber sections.

The chamber sections 15a, 15b are each provided with a takeup space 17 for collecting any penetrating water 18.

The partition walls 14 of the chamber 15 are provided with connecting openings 15d which connect the chamber sections 15a, 15b etc. successively and with one another (like a strand of pearls). The connecting openings are disposed spaced apart from the bottom of the pertaining chambers or chamber sections so as to provide suitable takeup spaces. The partition walls show connecting openings disposed so that they are not aligned but disposed laterally and/or vertically offset. Preferably each of the connecting openings is disposed spaced apart from the bottom of the pertaining takeup space.

On the whole, this provides two or more interconnected chambers or chamber sections and with the pertaining air guide in-between, a labyrinth seal 14 which is a particularly reliable protection of the barometric pressure sensor 20 from penetrating water. For maintenance work or following each trip the air guide may be completely or partially cleaned. The measuring probe 4 may for example be demounted and flushed and dried and/or purged by (in particular oil-free) compressed air.

FIG. 5 shows a section of an air duct 11, 12 or 13, with a water droplet 18 exemplarily inserted in the air duct. The interior of the air ducts shows a diameter or cross section 19. The diameter 19 is in particular between 0.5 mm and 2 mm. In this specific example the clear diameter 19 is 1 mm. The outer opening 6 shows a dimension 8 which is preferably smaller than the clear diameter 19. The diameter 8 of the outer opening is preferably 0.8 mm.

The dimensions 8 and 19 are matched to one another and to the properties of water so that any penetrating water forms a water plug 18 in the interior of an air duct, as is shown in FIG. 5. The plug can enter into the duct only far enough for establishing a balance of the force generated by the compressed air volume and the force caused by the total pressure. The smaller diameters considerably contribute to sealing.

The FIGS. 6 to 9 illustrate a more concrete exemplary embodiment of the measuring probe 4 including a probe body 5. FIG. 6 shows a perspective illustration with one of the air guides 10 drawn in dashed lines in the interior of the probe body 5 to provide a schematic overview. The front end shows the outwardly opening 6 at the tip of the probe body 5. The supply duct 11 follows as an air duct. In a central region a labyrinth seal 14 is comprised following in the rear region of the sensor duct 12 as an air duct.

FIG. 7 shows a side view and FIG. 8, section A-A from FIG. 7. FIG. 7 shows two of the total of e.g. four openings 6a (alternately, three or five or six or more openings are also conceivable) on the lateral circumference of the probe body 5, through which the static pressure is absorbed. The rear end of the probe body 5 then preferably shows a differential pressure sensor which captures a differential pressure of the total pressure and the local static pressure averaged over the circumference of the probe body 5 (ambient pressure locally averaged over the circumference of the probe body). It is also possible to employ two separate barometric pressure sensors used for determining the stagnation pressure.

FIG. 8 shows the opening 6 at the front tip.

FIG. 9 shows a cross section of the probe body 5 where it can be seen that the air guide 10 extends in the interior of the probe body 5 and presently comprises two chambers 15, 16, each showing chamber sections 15a and 15b, thus a total of four chambers (chamber sections). Each of the chambers 15, 16 is approximately “H” shaped in cross section with the supply provided through the supply duct 11 at the top end of the “H”. The connections with the second chamber 16 and the sensor duct 12 each also start at the top end of the chambers 15, 16. The chamber sections 15a, 15b are interconnected in a middle to top region via a connecting opening 15d. In this case the connecting opening 15d may also be referred to as an intermediate duct. The construction allows to use the lower legs of the “H”-shaped chambers 15, 16 as takeup spaces for any penetrating water 18.

The yaw sensor 30 is structured accordingly, comprising a probe body showing two openings and two air guides and preferably a differential pressure sensor 30c or two pressure sensors for obtaining a value of the differential pressure.

FIG. 10 shows a diagram with the pressure distribution around the surface of an object while air is streaming onto the object from the front. FIG. 10 shows a cross section of an aircraft wing but basically the pressure onto a surface depends on the angle of incidence and the properties of the object in other objects as well. While this object is drawn in a solid line, dashed lines show the pressure coefficient which is representative of the pressure acting locally on the surface of the object.

FIG. 10 shows what is known per se, that the local pressure onto the surface of an object is dependent on the position on the surface of the object. Thus, the local pressure may be higher or lower than the normal, inactive ambient pressure (and further also depends on the air speed).

The dependence on the position is a problem if a vehicle moving relative to the ambient air—such as a bicycle—is to capture the ambient pressure. Even the interior of an object does not show the normal ambient pressure but the pressure is influenced by the traveling speed, the wind speed, the wind direction and also by the structure of the object.

If an aerodynamic drag coefficient of a bicycle is to be obtained, the sensor values required must be captured as precisely as possible. It is a great advantage if the gradient of a path and/or also the wind direction are captured as precisely as possible.

FIG. 11 exemplarily shows the “ambient pressure” measured directly with the barometric pressure sensor 20 by means of a bicycle component 1, over the air speed respectively the speed of the bicycle relative to the wind. The pressure is plotted in Newton per square meter (N/m² or Pa) over the speed in kilometers per hour. This specific case shows that the curve of the air pressure signals 21, measured at actually the same ambient pressure, strongly depends on the relative speed. With the air speed increasing, the measured air pressure signal 21 decreases. The difference in the illustrated speed range of 0 to 100 km/h is approximately 10 mbar or 1000 Pa.

The concrete curve depends on the arrangement of the barometric pressure sensor for measuring the ambient pressure, on the precise configuration of the measuring device respectively the bicycle component 1 and also on the wind direction. The effect cannot be generally avoided, independently of a selected position. Even if, as in this case, the barometric pressure sensor 20 for the ambient pressure is disposed inside the housing 3, the relative wind blast and relative direction of the air may impair the measuring quality. Dynamic effects may show, which increase or decrease the measured value. The air may stagnate in front of the sensor inlet or Bernoulli's theorem may show a measured pressure value 21 that is lower than the true ambient pressure.

The bicycle component 1 comprises in the computer memory 51, calibration data 53 which allow, based on measurement data or empirical data, to correct the air pressure signal 21 first captured by a barometric pressure sensor 20. Using the stagnation pressure values 26 captured with the stagnation pressure sensor system 25 is most advantageous. The result may be further improved, taking into account the yaw angle 32 captured with the yaw sensor 30.

This enables considerable improvement to the determination of the current elevation of the racing bicycle 100, and the gradient or the gradient angle 201 of a path 200 can be derived at considerably improved accuracy.

Since the inclination angle or the gradient or the slope of a path considerably influences the driving power required, an aerodynamic drag coefficient can thus be determined at considerably improved accuracy. A negative gradient tends to be called slope. In a slope the aerodynamic drag coefficient also exerts a big influence.

Furthermore, the rolling resistance also influences the power required. To this end, further measurement values may be captured and analysed or values captured previously are used. The rolling resistance is influenced by the tires used, the tire pressure, the weight of the bicycle and of the rider and the road condition, and may be obtained, computed, and/or estimated.

Data may be captured and evaluated to obtain pertaining calibration data 53 either in a wind tunnel or on suitable roads, given suitable ambient and wind conditions. The calibration data 53 may then be used in normal operation to increase the accuracy of the measurement results and the derived values. The calibration data 53 for the calibrating matrix is derived either from tests in the wind tunnel or from road tests with no variations of elevation in one range of air speeds and yaw angles.

The FIGS. 12 to 14 show the elevation curve of a path 200 over the track and values measured and derived during riding on said track.

In FIG. 12 the solid line shows the actual elevation curve 202 of the path 200 in meters (relative to the starting point) over the illustrated length in meters. Cross marks indicate single measuring points obtained and recorded during the ride over the illustrated track. The inserted measurement values are elevation signals 36 obtained by a satellite sensor 35.

For this purpose, for example, a GPS sensor or another satellite sensor 35 of a global navigation satellite system (GNSS) may be used. Also possible are systems using pseudosatellites providing a local satellite system and enabling triangulation of the elevation and/or position.

One can clearly see the high accuracy of the satellite sensor 35 and the elevation signals 36. One can also directly recognize that the resolution of the elevation signals 36 of the satellite sensor 35 is comparatively coarse. Elevation differences of just under 2 m are recognized. A resolution of 2 m is not sufficient for computing a gradient for determining an aerodynamic drag coefficient when operating a bicycle.

Therefore, using only satellite sensors 35 for obtaining a local gradient does not yield satisfactory results for example if an aerodynamic drag coefficient is to be computed therefrom. An interpolation between each of the measurement values does not provide the required accuracy either since the elevation curves of many paths considerably differ from the particularly simple test track shown. Thus the gradient may considerably change locally already over one meter or over a few meters.

FIG. 12 additionally shows in a broken line the curve of the measured ambient pressure during a disruptive event. These kinds of interference signals 29 may appear due to a passing vehicle and in particular a passing truck. Then the measured ambient pressure and the elevation computed therefrom considerably deviates from the true height. These events may be discounted through internal filtering. Preceding and following values are captured and taken into account and offset against the elevation determination through GPS. The typical curve with alternating pressure peaks and pressure minima facilitates filtering. Filtering also allows to prevent miscalculation of the wind direction and wind speed.

FIG. 13 shows the same track as does FIG. 12 wherein on the one hand, the actual elevation curve 202 of the test track is plotted and on the other hand, an elevation profile derived from an air pressure signal 21 of a barometric pressure sensor 20.

Initially a reference value 28 is captured which is then used for determining an elevation difference. It can be seen that as measuring begins, the curve measured by the barometric pressure sensors shows a close match with the actual elevation curve 202. Around the middle the difference in elevation is already nearly 2 m at the value 23.

As the track continues, the elevation curve measured with the barometric pressure sensor 20 shows a systematic offset or divergence versus the actual elevation curve 202. The reason is that the barometric pressure sensor 20 does not capture the actual elevation but a measure of the ambient pressure. Although the absolute ambient pressure also depends on the elevation, it may vary e.g. due to the weather. Now if the air pressure drops during the ride on the track or if the air pressure rises, then the values so determined may diverge. This is shown exemplarily by the value 27. Again the result is that the values are not sufficiently precise for obtaining a high quality, aerodynamic drag coefficient. For this, a higher accuracy of capturing the elevation is useful.

Finally, FIG. 14 in turn shows on the one hand, the actual elevation curve 202 of the test track and on the other hand, a curve of the elevation values 24 corrected via the various sensors respectively the measurement results of the various sensors.

To this end the air pressure signals 21 of the barometric pressure sensor 20 disposed in the interior of the housing 3 of the measuring device 2 are corrected according to the calibration data 52, by way of the stagnation pressure values 26 captured by the stagnation pressure sensor system 25 and the yaw angle values 31 captured by the yaw sensor 30, according to the basic principle of the illustration in FIG. 11, to obtain a largely correct measure of the current elevation value 24.

Moreover, in addition to capturing the air pressure signals 21, the satellite sensor 35 is also employed for determining elevation measures. At periodic intervals the high-precision satellite sensor 35 is employed to obtain a comparison value. If the elevation value 24 obtained by way of the various barometric pressure sensors 20, 25 and 30 significantly deviates from the elevation signal 36 of the satellite sensor 35, a new reference signal 28 is derived so that an accurately corrected elevation value 24 ensues with the pertaining air pressure signal 21. To avoid recalibration owing to noisy measurement values, corrections only take place if differences show over a significant period of time.

This method combines the advantages of the high accuracy of satellite sensors 35 with the advantages of the high resolution of barometric pressure sensors 21. At the same time the drawbacks of the coarse resolution of satellite sensors 35 and of the conceivable air pressure fluctuations from barometric pressure sensors 20 are avoided. As can be seen in FIG. 14, the result is high congruence of the effective curve of the elevation values 24 with the actual elevation curve 202 of the test track.

A (first) reference signal 28 may for example be input or captured at the start of a ride or when the elevation is known. Differencing of the air pressure signal 21 during riding and the reference signal 28 allows to obtain a measure of the current elevation. The reference signal 28 may firstly be obtained by obtaining an initial air pressure signal 21 which is used as a reference signal 28 for following measurements. The pertaining reference signal 28 may also be input or captured by the satellite sensor 35. During the ride the reference signal 28 may be updated periodically and at irregular time intervals.

Corrections of the reference signal 28 used for computing a measure of elevation 23 may be carried out for example if the sum total of the deviations between the elevation signals 36 of the satellite sensor 35 and the obtained elevation values 24 exceeds a specified measure or a specified threshold over a given time period. For example, a mean value may be computed over a specific distance or after a specific time period, which is then used for comparison.

Elevation signals 36 are preferably measured between approximately 20 and 30 times per second and approximately 3 to 5 times per minute, in particular at a frequency of approximately 0.1 Hz. The frequency at which a current measure of elevation is captured from a current, characteristic air pressure signal for the ambient pressure is preferably higher and is in particular between 0.1 Hz and 1 kHz and preferably between 1 Hz and 100 Hz, particularly preferably approximately 50 Hz.

Particularly preferably, the ratio of the measuring frequency of the air pressure signal 21 for the ambient pressure to the measuring frequency of an elevation signal 36 is larger than 10 and in particular larger than 100 and preferably smaller than 5000. This allows to achieve a high measure of accuracy while energy demand remains low.

FIG. 14 additionally illustrates three curves 41, 42 and 43 of the measuring frequencies. The measuring frequency for capturing the signals and in particular capturing the elevation signals or capturing the air pressure signals is dependent on the currently prevailing riding conditions and may be adjusted by means of the control device 40 and modified as needed. Thus the measuring frequency is set higher in particular in gradients and particularly preferably in slopes, than on straight tracks. The curves 41 to 43 each show the measuring frequency over the distance and they are shown vertically offset for better clarity, to illustrate each curve separately.

The first measuring curve 41 shows an example of a basically constant measuring frequency, where the state of the energy supply drops beneath a threshold approximately in the middle of the distance. Then, energy saving measures are initiated and the measuring frequency is clearly reduced. It is possible that at the reduced level the measuring frequency is still varied in dependence on the current riding conditions, for example it increases as the speed increases or in the case of gradients or slopes. In the plane the measuring frequency can be reduced still further.

The second measuring curve 42 shows a control variant where an increased measuring frequency is set in the region of the first gradient. As the middle plateau is reached, the measuring frequency is considerably reduced in what is now a plane level (e. g. factor ½). As a slope begins, the measuring frequency is greatly increased so as to achieve a very high precision for the higher riding speed downhill.

The third measuring curve 43 shows an example where in the region of the inclinations of the path (gradient/slope) the measuring frequency is increased, while the measuring frequency is reduced in the plane. This achieves increased precision in the region of the inclinations and energy demand is reduced in the plane. The curves 41 to 43 may in particular show not only the measuring frequency over the track but may also show curves of the measuring frequency over the riding time.

The schematically shown curves 41 to 43 show the measuring frequency over the track for a constant riding speed. The curves bend accordingly in the case of different riding speeds.

Preferably, the measuring curves 41 to 43 each show identical measuring frequencies at the start, at the time 0. The absolute elevation is shown at an offset to better distinguish the curves graphically.

Furthermore, FIG. 14 shows the gradient curve 44 over the measuring distance in a dash-dotted line. At the start the curve of the gradient over the first third of the measuring distance shows a constant level. The gradient shows a value (scale on the right) of +10.0. In the second third in the plane the gradient is 0.0, and in the last third there is a slope with a gradient of −10.0. A gradient value 201 may be derived through the periodically captured air pressure signals 21 and the associated track data. To this end the data are first averaged and filtered.

The known weight of the bicycle and the rider allow to derive performance data from the current gradient value and the current speed value. It is taken into account whether and how the bicycle is accelerated.

Taking into account the input performance e.g. via force sensors on the pedals or torque sensors in suitable positions, all of the data allows conclusions about the currently prevailing aerodynamic drag. This assists the rider in taking, and maintaining, an optimal position during riding, since the relevant values are periodically re-captured and displayed. Computation is in particular done at a frequency of a minimum of 5 times per minute, preferably at least 20 times per minute. Frequencies of 0.5 Hz or 1 Hz or 10 Hz or more are likewise conceivable.

On the whole, an advantageous bicycle component and an advantageous method are disclosed which enable improved options for measuring data in a bicycle. Depending on the positioning of a barometric pressure sensor for obtaining the absolute ambient pressure, the measurement result is influenced by the speed of the bicycle, the wind speed and the wind direction, and can thus provide results which are firstly imprecise. If the stagnation pressure is measured using for example barometric pressure sensors with a pitot tube open to the front in the traveling direction, a total pressure will ensue which depends on the absolutely prevailing air pressure in the ambience and on the traveling speed. This pressure signal is not alone sufficient for determining an elevation or gradient since an impression of a gradient would show if the rider accelerates in a plane.

If the barometric pressure sensor for obtaining the absolute ambient air pressure is located for example in the housing of the bicycle component or in the measuring device 2, then the air stagnates in front of the housing as a consequence of the wind blast or the traveling speed and at the front tip of the housing generates a total pressure which negatively (or also positively) influences the absolute air pressure measured in the interior of the housing.

If the barometric pressure sensor for capturing the absolute air pressure is disposed on a side of the housing next to an opening, then the result again shows a negative influence due to Bernoulli's theorem. Then, the air flowing past may generate an underpressure which would again—depending on the speed—show a negative influence on the absolute pressure.

This is why correction of the air pressure signal 21 by the stagnation pressure value 26 is useful and advantageous if the bicycle component 1 is to obtain minor and also tiny gradients. The correction is in particular done together with a calibrating matrix captured in previous tests under known conditions. Calibration values are in particular captured and stored for variations of the relative speed and/or variations of the yaw angle. A correction is for example advantageous and important to sufficiently precisely obtain the air drag.

The correction of an elevation value 24 by means of an elevation signal 36 of a satellite sensor is advantageous since in circuits the bicycle component shows the same elevation at the end as at the beginning of the circuit.

Due to the relatively large graduation in measuring, an elevation profile is as a rule captured via barometric pressure sensors. However, known bicycle computers tend to show different elevation data at the beginning and the end of a circuit due to air pressure fluctuations. In fact, the rider has traveled a complete round and at the end of the round he is located at precisely the same elevation as he was at the beginning of the round.

The presently disclosed combination of evaluations of satellite sensors and pressure sensors allows a very precise elevation determination and in particular a very precise determination of the gradient of a path or a track. Since the power required for driving the bicycle is considerably dependent on the acceleration, the gradient if any, the rolling resistance, and the air drag, high accuracy can thus be achieved.

The invention allows the rider to also measure and evaluate during riding, his seated position as well as the bicycle components and other equipment such as his helmet, suit, clothing etc. Thus the rider may find out what for him is the optimal seated position and combination of bicycle parts and equipment and determine what for him is e.g. the best helmet in terms of aerodynamics offering the lowest air drag in his preferred position.

Other than the options described for calibrating the barometric pressure sensor during rides, re-calibration can also be performed if the barometric pressure sensor found a specific gradient or a specific slope. For example, following a gradient or a slope of 5 m or 10 m. Re-calibration can also be performed at specific time intervals. Also, a combination of calibration based on time and exceeded elevation differences may be performed.

Air pressure values are preferably measured using barometric pressure sensors showing a measuring range encompassing at least 25% and in particular at least 50% of the normal pressure of (approximately) 100 kPa. For capturing the ambient pressure or the total pressure, barometric pressure sensors are preferred showing a measuring range of higher than 30 kPa and in particular at least 50 kPa or 60 kPa or 80 kPa.

In preferred configurations, the measuring range of the differential pressure sensors employed is smaller than that of the barometric pressure sensors employed. Differential pressure sensors are in particular employed for capturing the stagnation pressure and/or the yaw angles. The measuring range of a differential pressure sensor employed is preferably less than 20 kPa and in particular less than 10 kPa and particularly preferably less than 5 kPa or 2 kPa or 1 kPa. In a specific example, differential pressure sensors are used showing a measuring range of 0.5 kPa (+/−20%). This enables a high resolution and accuracy.

The measuring range of a barometric pressure sensor for capturing the ambient pressure or the total pressure is preferably larger than the measuring range of a differential pressure sensor for the stagnation pressure or for determining the yaw angle.

The ratio of the measuring range of a barometric pressure sensor for capturing the ambient pressure or the total pressure to the measuring range of a differential pressure sensor for stagnation pressure or for determining the yaw angle is preferably higher than 5:1 and in particular higher than 10:1 and particularly preferably higher than 50:1.

In all the configurations, it is preferred to perform temperature compensation of the measurement values to prevent thermal effects.

The configuration of the measuring probe respectively probe body 5 is advantageous since it allows operating a bicycle independently of the external conditions. The configuration of the air guide in the interior of the probe body 5 reliably prevents any penetrating water from being conducted toward a barometric pressure sensor. And, in case that a droplet of water or dirt has in fact entered, it is retained in the takeup space 17 of a chamber 15. Thereafter the water may exit for example by evaporation, or manual cleaning, flushing and/or purging is performed after removing the probe body 5, which is in particular clipped on. The air ducts and their dimensions and the chamber(s) provide a labyrinth seal with an additional takeup space so that the measuring probe 4 is waterproof under any conditions expected in everyday use.

A conventional membrane in the interior of the measuring probe for mechanically separating the supply duct 11 from the sensor duct 12 achieves sufficient tightness as a rule. There is the drawback that accuracy is considerably reduced and the measurement results are thus deteriorated so that an aerodynamic drag coefficient cannot be determined with sufficient accuracy. The measuring probe 4 presently disclosed achieves sufficient tightness and sufficient accuracy.

Preferably, the probe body 5 is manufactured by way of 3D printing, at least partially or entirely of plastic, and/or at least partially or entirely of metal. The interior may show an integral seal or labyrinth seal. 3D printing allows much greater ease of manufacturing a probe body than conventional technology does. Thus, hollow spaces may be provided in places where solid material is otherwise required for reasons of process technology.

### LIST OF REFERENCE NUMERALS

| | |
|---|---|
| 1 | bicycle component |
| 2 | measuring device |
| 3 | housing |
| 4 | measuring probe |
| 5 | probe body |
| 6 | opening in 5 |
| 6a | opening |
| 7 | opening |
| 8 | dimension of 5 |
| 10 | air guide |
| 11 | air duct, supply duct |
| 12 | air duct, sensor duct |
| 13 | air duct, intermediate duct |
| 14 | labyrinth seal |
| 15 | chamber |
| 15a | chamber section |
| 15b | chamber section |
| 15c | partition wall |
| 15d | connecting opening |
| 16 | chamber |
| 17 | takeup space in 15, 16 |
| 18 | water |
| 19 | cross section of 11-13 |
| 20 | barometric pressure sensor, absolute pressure transducer |
| 21 | air pressure signal of 20, sensor value of 20 |
| 22 | corrected ambient pressure value |
| 23 | measure of elevation |
| 24 | elevation value |
| 25 | stagnation pressure sensor system, pitot sensor |
| 25c | differential pressure sensor |
| 26 | stagnation pressure value, sensor value of 25 |
| 27 | change of elevation |
| 28 | reference signal |
| 29 | interference signal |
| 30 | yaw sensor system |
| 30a | opening |
| 30b | opening |
| 30c | differential pressure sensor |
| 32 | yaw angle |
| 33 | relative wind direction and wind force |
| 34 | traveling speed |
| 35 | satellite sensor |
| 36 | elevation signal |
| 37 | humidity sensor |
| 38 | acceleration sensor |
| 40 | control device |
| 41 | first measuring curve |
| 42 | second measuring curve |
| 43 | third measuring curve |
| 44 | gradient curve |
| 50 | computer |
| 51 | memory |
| 52 | data interface, network interface |
| 53 | calibration data |
| 54 | energy source |
| 100 | bicycle |
| 101 | wheel, front wheel |
| 102 | wheel, rear wheel |
| 103 | frame |
| 104 | fork, suspension fork |
| 106 | handlebar |
| 107 | saddle |
| 109 | spoke |
| 110 | rim |
| 112 | pedal crank |
| 115 | speed sensor |
| 116 | power sensor, force sensor |
| 120 | elevation |
| 200 | path |
| 201 | gradient value, gradient angle |
| 202 | elevation curve |
| 300 | satellite system |
| 301 | satellite |

1. A bicycle component, comprising: a measuring device with a housing and at least one measuring probe connected therewith; having at least one unheated probe body having at least one exterior opening; the exterior opening being connected through an air guide with at least one barometric pressure sensor disposed remote from the exterior opening; wherein the air guide comprises at least two air ducts and at least one internal chamber connected with at least two air ducts; wherein one of the air ducts is configured as a supply duct and begins at the exterior opening; wherein another air duct serves as a sensor duct and connects the internal chamber with the barometric pressure sensor; and wherein at least one of the air ducts has a minimum clear diameter of less than 2.5 mm.

2. The bicycle component according to claim 1, wherein at least one of the air ducts has a diameter of less than 2 mm at the exterior opening.

3. The bicycle component according to claim 1, wherein the internal chamber is configured with at least one takeup space for any penetrated water.

4. The bicycle component according claim 3, wherein at least one takeup space is configured well-shaped.

5. The bicycle component according to claim 1, wherein the internal chamber comprises at least one partition wall which subdivides the internal chamber into at least two chamber sections, and wherein the two chamber sections are interconnected through at least one connecting opening.

6. The bicycle component according to claim 5, wherein in use as intended the connecting opening is disposed spaced apart from the bottom end of the internal chamber.

7. The bicycle component according to claim 1, wherein the air guide is configured with at least one type of labyrinth seal.

8. The bicycle component according to claim 7, wherein the labyrinth seal comprises two or more internal chambers and disposed in-between, air ducts.

9. The bicycle component according to claim 1, wherein the housing and/or the probe body is configured aerodynamically.

10. The bicycle component according to the claim 1, wherein the probe body is configured elongated and has a substantially rotationally symmetrical outer surface.

11. The bicycle component according to claim 1, wherein the probe body consists at least considerably of at least one metal.

12. The bicycle component according to claim 1, wherein at least a substantial part of the probe body is manufactured by way of 3D printing, forming the air guide.

13. The bicycle component according to claim 1, wherein the probe body comprises at least two or three openings, to which at least one barometric pressure sensor is assigned.

14. The bicycle component according to claim 1, wherein the probe body is plugged onto the housing.

15. The bicycle component according to claim 1, wherein the at least one barometric pressure sensor is disposed in the housing.

16. The bicycle component according to claim 1, wherein a computer is disposed in the housing.

17. The bicycle component according to claim 1, wherein the dimensions of the exterior openings and of the internal air guide are designed such that the resulting cross section of the air ducts is small enough for the water surface tension at the cross section of the air duct to withstand the force ensuing from the dynamic air pressure with air speeds of up to 35 m/s, and thus to prevent water from penetrating up to the barometric pressure sensor.

18. The bicycle component according to claim 1, wherein each internal chamber contains a takeup space, in which water entering into the measuring probe through the exterior openings may collect, and is positioned such that it is remote from the outlet of the internal chamber in the direction of the barometric pressure sensor.
